(19) United States
(12) Patent Application Publication
(10) Pub. No.: US 2024/0017054 A1
(43) Pub. Date: Jan. 18, 2024

(54) ALIGNMENT GARMENT FOR USE WITH A FULLY IMPLANTABLE SYSTEM

(71) Applicant: Medtronic, Inc., Minneapolis, MN (US)

(72) Inventors: Stephen M. Nelson, Plymouth, MN (US); David J. Peichel, Minneapolis, MN (US); Eric A. Schilling, Ham Lake, MN (US); Nathanael O. Mason, Holliston, MA (US); Robert Scadding Moir, Cambridge (GB); Lillian E. Clarke, Cambridge (GB); Ismail Akram, Cambridge (GB); Glenn Le Faou, Hertfordshire (GB); Richard James Andrews, Cambridge (GB); Jordi Vilanova Hanusaik, Cambridge (GB); Anna Rebecca Louise Wilson, Cambridge (GB); Arron Carmo do Rosario Rodrigues, Cambridge (GB)

(21) Appl. No.: 18/251,359

(22) PCT Filed: Sep. 17, 2021
(86) PCT No.: PCT/US2021/050824

§ 371 Date: May 1, 2023

Related U.S. Application Data
(60) Provisional application No. 63/108,621, filed on Nov. 2, 2020.

(51) Int. Cl. A61M 60/875; A61M 60/178; A61M 60/232; H02J 50/10

(52) U.S. Cl. CPC A61M 60/875; A61M 60/178; A61M 60/232; H02J 50/10; A61M 2209/088; A61M 2205/8243; H02J 2310/23

(57) ABSTRACT

An alignment vest for holding an external coil in a predetermined location. The alignment vest may comprise at least one attachment mechanism for holding the external coil in the predetermined location. The alignment vest may also comprise a panel having a fastening mechanism and a plurality of straps, the front panel being secured to at least one of the plurality of straps.

### FIELD

The present technology is generally related to an alignment system for use with an implantable medical device system that has the transfer of energy from a power source outside the body to an implanted medical device inside the body.

### BACKGROUND

Mechanical circulatory support devices, including ventricular assist devices (“VADs”), are used for people that have weakened hearts and/or heart failure. VADs can aid the heart's natural pumping to increase the amount of blood flow throughout the body. VADs require various external and internal components to work together to run the system so that blood is received from the patient's heart and the VAD pumps the blood back into arterial circulation. When the external and internal components of the VAD work together properly, an individual with a weakened heart and/or advanced heart failure can lead a more active and fuller life.

A transcutaneous energy transfer system (“TETS”) may be used to power implantable devices such as the VADs. Generally, a TETS can transfer energy from an external transmission coil to a receiving coil that is implanted under the skin. A TETS may be used to supplement, replace, or charge an implanted power source such as a rechargeable battery.

Some of the components that are used in the VADs include an external transmission coil and an implanted receiving coil. Various components must be properly aligned to prevent an interruption of the operation of the VADs. Movements of a patient can cause the misalignment of components from the VADs and/or the TETS which may cause the system to malfunction and in certain instances fail when power is not being transferred into the system.

### SUMMARY

The techniques of this disclosure generally relate to an alignment system for a wireless energy transfer system, including wearable medical devices that use a transcutaneous energy transfer system (“TETS”), that require the alignment of certain external and internal components to continually facilitate the effective transfer of energy. In one aspect, an alignment vest for holding an external coil in a predetermined location, the alignment vest comprising: at least one attachment mechanism for holding the external coil in the predetermined location; a panel secured to the attachment mechanism; and a plurality of straps, the front panel being secured to at least one of the plurality of straps.

In another aspect, the two attachment mechanisms are secured to at least a portion of the panel.

In another aspect, the alignment vest may further comprise a first band, a second band, a third band, and a fourth band, the first band, the second band, the third band and the fourth band each being pretensioned bands that are configured to secure the first attachment mechanism to the panel.

In another aspect, the alignment vest may further comprise a fifth band, a sixth band, a seventh band, and an eighth band, the fifth band, the sixth band, the seventh band, and the eighth band each being pretensioned bands that are configured to secure the second attachment mechanism to the panel.

In another aspect, the panel further comprises a plurality of portions and a fastening mechanism, the fastening mechanism being a zipper and the plurality of portions being releasably secured together with the zipper.

In another aspect, the plurality of straps includes a first strap, a second strap, a third strap, and a fourth strap, at least a portion of each strap being secured to a portion of the panel.

In another aspect, each strap from the plurality of straps has at least one slider, the at least one slider being configured to adjust the length of the strap.

In another aspect, the at least one slider is made from a non-conductive material.

In another aspect, at least a portion of the first strap, the second strap, the third strap, and the fourth strap are secured to a retaining mechanism.

In one aspect, an alignment vest for holding an external coil in a predetermined location, the alignment vest may comprise: at least one attachment mechanism for holding the external coil in the predetermined location; a panel having a fastening mechanism and a first band, a second band, a third band, and a fourth band, the first band, the second band, the third band and the forth band each being pretensioned bands that are configured to secure the at least one attachment mechanism to the panel; and a plurality of straps, the panel being secured to at least one of the plurality of straps.

In one aspect, the alignment vest further comprises a second attachment mechanism and a fifth band, a sixth band, a seventh band and an eighth band, the fifth band, the sixth band, the seventh band and the eighth band each being pretensioned bands that are configured to secure the at second attachment mechanism to the panel.

In another aspect, the fastening mechanism is at least one of a zipper and a swan hook.

In another aspect, the panel has at least two portions, the at least two portions being releasably secured together with the zipper.

In another aspect, the zipper is made from a non-conductive material.

In another aspect, each strap in the plurality of straps has at least one slider, the at least one slider being configured to adjust the length of each strap in the plurality of straps.

In another aspect, the at least one slider is made from a non-conductive material.

In one aspect, an alignment vest for holding an external coil in a predetermined location, the alignment vest may comprise: a panel having a fastening mechanism; a first attachment mechanism for holding the external coil in a first predetermined location, the first attachment mechanism being secured to a portion of the panel; a second attachment mechanism for holding the external coil in a second predetermined location, the second attachment mechanism being secured to a portion of the panel; a first strap, a second strap, a third strap and a fourth strap, at least a portion of the panel being secured to at least a portion of the first strap, the second strap, the third strap and the fourth strap.

In another aspect, the first strap is between approximately 2-10 inches in length and the second strap is between approximately 2-10 inches in length.

In another aspect, the fastening mechanism is a zipper, the zipper having a length of between 5-15 inches in length.

In another aspect, the zipper is made from non-conductive material.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a front inside of the body view of a patient with a left ventricular assist device, receiving coil, and controller fully implanted within the patient;

FIG. 2 is a front outside of the body view of the patient shown in FIG. 1 showing a battery and transmission coil coupled to the patient;

FIG. 3 is a front view of the receiving coil and controller shown in FIG. 1;

FIG. 4 is a front view of an exemplary embodiment of the alignment system for the pectoral or axilla region of the patient's body with a vest on the body of the patient;

FIG. 5 is a rear view of the exemplary embodiment of the alignment system for the pectoral or axilla region of the patient's body with the vest on the body of the patient shown in FIG. 4;

FIG. 6 is a front view of an exemplary embodiment of the alignment system for the pectoral or axilla region of the patient's body with the vest on the body of the patient; and

FIG. 7 is a rear view of the exemplary embodiment of the alignment system for the pectoral or axilla region of the patient's body with the vest on the body of the patient in FIG. 6.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

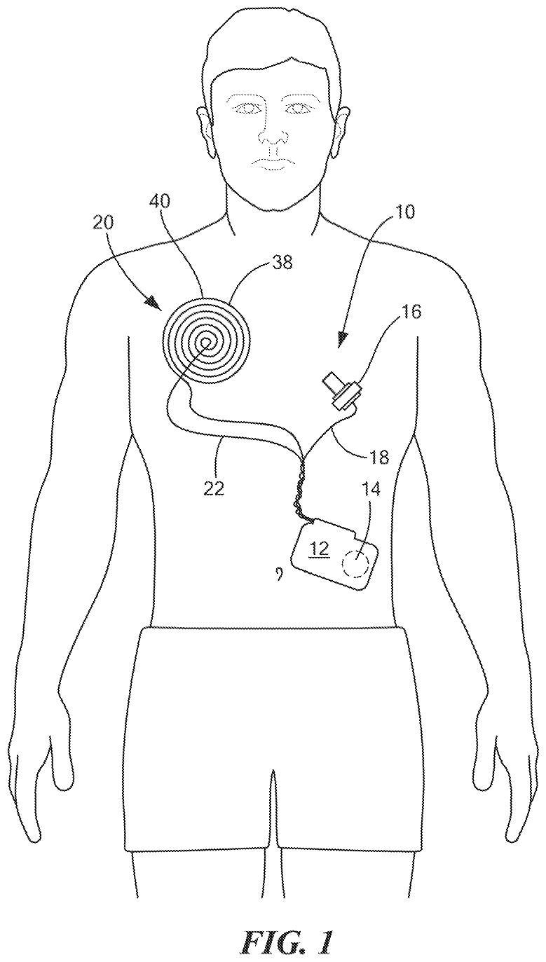

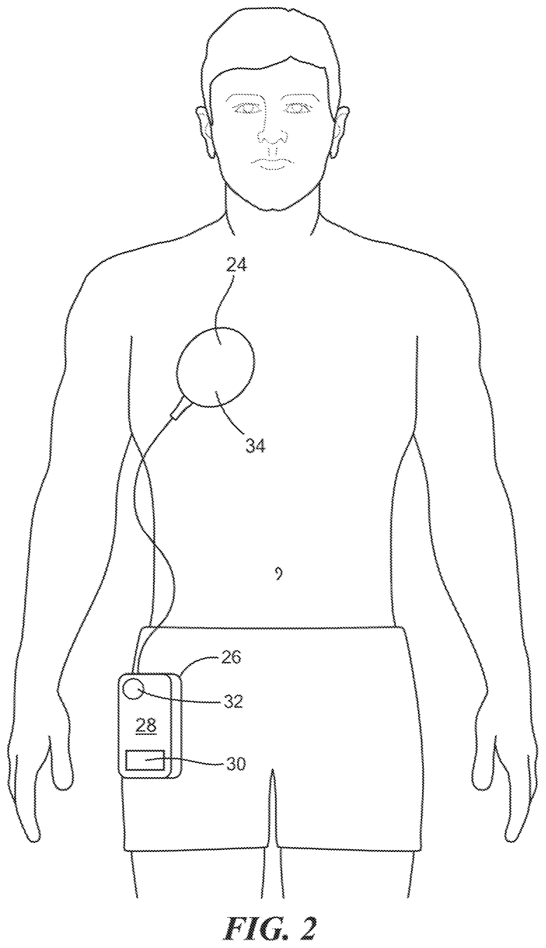

Referring now to the drawings in which like reference designators refer to like elements there is shown in FIGS. 1 and 2 an exemplary transcutaneous energy transfer system (“TETS”) constructed in accordance with the principles of the present application and designated generally as “10.” While there is discussion about a particular TETS, it will be understood that the TETS can be used for wireless energy transfer for another type of implantable medical device and can be in a variety of different configurations. The TETS 10 may be fully implantable within a patient, whether human or animal, which is to say there are no percutaneous connections between the implanted components of the TETS 10 and the components outside of the body of the patient. In the configuration shown in FIG. 1, the TETS 10 includes an internal controller 12 implanted within the body of the patient. The internal controller 12 may include a power source 14 configured to power the components of the controller and provide power to one or more implantable medical devices, for example, an implantable blood pump such as a ventricular assist device system (“VADs”) 16 implanted within the left ventricle of the patient's heart. The power source 14 may include a variety of different types of power sources including an implantable battery. The VADs 16 may include centrifugal pumps, axial pumps, or other kinds electromagnetic pumps configured to pump blood from the heart to blood vessels to circulate around the body. One such centrifugal pump is the HVAD and is shown and described in U.S. Pat. No. 7,997,854, the entirety of which is incorporated by reference. One such axial pump is the MVAD and is shown and described in U.S. Pat. No. 8,419,609. In an exemplary configuration, the VADs 16 is electrically coupled to the internal controller 12 by one or more implanted conductors 18 configured to provide power to the VADs 16, relay one or more measured feedback signals from the VADs 16, and/or provide operating instructions to the VADs 16.

Continuing to refer to FIG. 1, a receiving coil 20 may also be coupled to the internal controller 12 by, for example, one or more implanted conductors 22. In an exemplary configuration, the receiving coil 20 may be implanted subcutaneously proximate the thoracic cavity, although any subcutaneous position may be utilized for implanting the receiving coil 20. The receiving coil 20 is configured to be inductively powered through the patient's skin by a transmission coil 24 (seen in FIG. 2) disposed opposite the receiving coil 20 on the outside/exterior of the patient's body. For example, as shown in FIG. 2, a transmission coil 24 may be coupled to an external controller 26 having a power source 28, for example, a portable battery carried by the patient. The transmission coil 24 may have raised portions or be a smooth surface. The raised portions may be configured to engage with different components of the alignment system. In one configuration, the battery is configured to generate a radiofrequency signal for transmission of energy from the transmission coil 24 to the receiving coil 20. The receiving coil 20 may be configured for transcutaneous inductive communication with the transmission coil 24.

The external controller 26 may further include a display 30 configured to provide information to the patient in one or more ways including audibly, visually, and/or tacitly. Additionally, the external controller 26 may further include processing circuitry 32 which can process and transmit information. The processing circuitry 32 may perform various simple and complex operations including, but not limited to, the amplification of signals, computations, and the movement of data from one place to another. The various simple and complex operations may be performed by the processing circuitry 32 in the external controller 26. The processing circuitry 32 may include one or more processors and memory. The external controller 26 may further comprise software, which is stored in, for example, memory at the external controller 26, or stored in external memory (e.g., database, storage array, network storage device, etc.) accessible by the external controller 26. The software may be executable by the processing circuitry 32. The software may include a client application operable to provide a service to a human or non-human user via the external controller 26, with the support of a host computer.

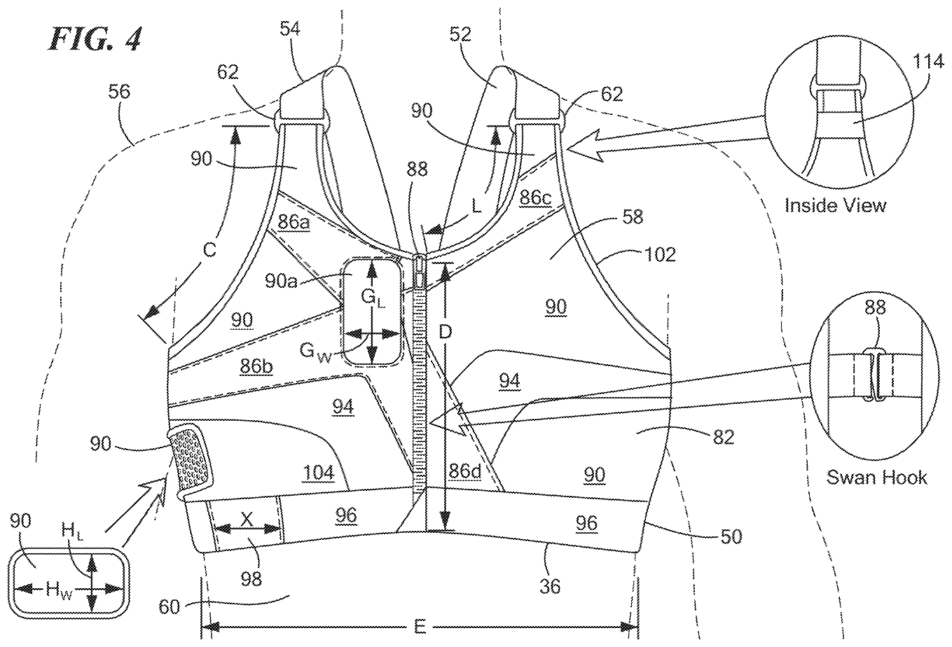

Referring back to FIG. 2, the transmission coil 24 is optionally housed within sealed packaging 34 to protect the transmission coil 24 and is optionally secured by a garment 36, discussed in more detail in FIGS. 4-7 that is configured to maintain the transmission coil 24 in a fixed position for power transmission to the receiving coil 20.

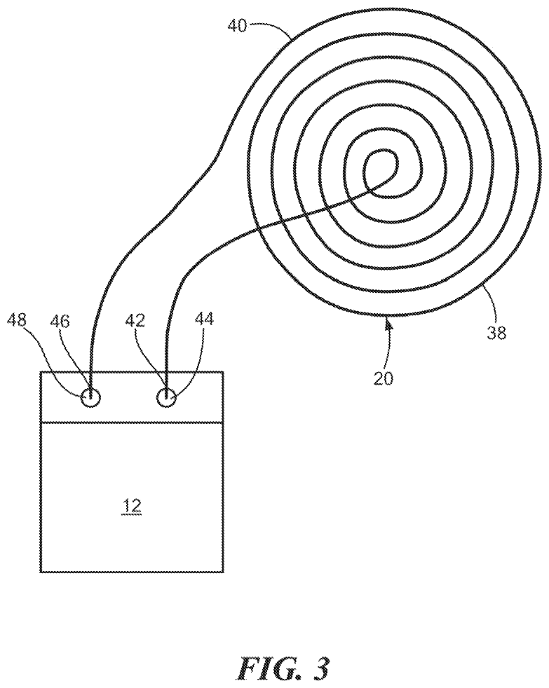

Referring now to FIGS. 1 and 3, the receiving coil 20 includes at least one conductor 38 defining a coil 40 and configured to power the VAD 16. The receiving coil 20 may be any shape or size and, for example, in one configuration, the receiving coil 20 may define a substantially planar coil defining a diameter such that is substantially co-planar with an interior surface of the dermis. The receiving coil 20 may be coupled with the internal controller 12 with a first end 42 being electrically coupled to a first coupling 44 of the internal controller 12 and a second end 46 being coupled to a second coupling 48 of the internal controller 12 such that a voltage may be applied to the coil 20. Alternatively, the receiving coil 20 may be coupled with the internal controller 12 using joints or any other configuration. Although not shown, temperature sensors may be found throughout the TETS 10.

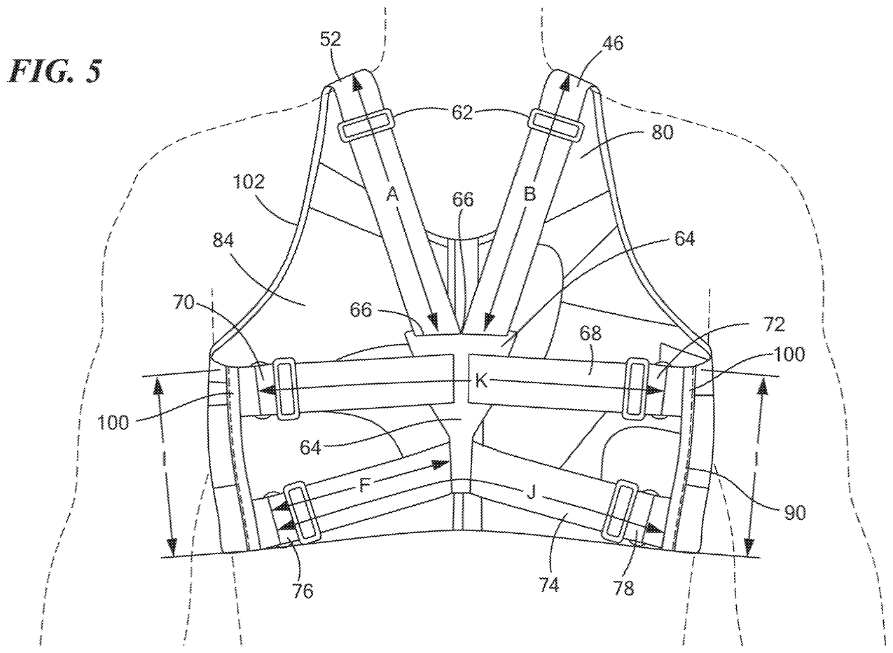

Referring to FIGS. 4-5 wherein an exemplary garment 36 is shown. The garment 36 may be used to help maintain the position and alignment of certain external and internal components of the wireless energy transfer system. As a non-limiting example as shown in FIG. 1, the implanted charging component may be the receiving coil 20. It will be understood that the implanted charging component may be any other implanted component but will be generally referred to in the application as the receiving coil 20. Also, the external charging component may be any external charging component, but as a non-limiting example as shown in FIG. 2, the external charging component may be the transmission coil 24. It will be understood that the external charging component may be any other external component but will be generally referred to the in application as the transmission coil 24. To allow for energy to transfer in the TETS, the transmission coil 24 and the receiving coil 20 must be properly aligned.

Continuing to refer to FIGS. 4-5, the garment 36 may be used to maintain the alignment of the external charging component/transmission coil 24 over the implanted charging component/receiving coil 20 within, for example, a 2 inch tolerance along the planar and linear axes of the components for the TETS system so that power can continually be transferred effectively and efficiently. In one embodiment, the garment 36 may be a vest 50. The vest configuration can have numerous different embodiments that a configured to fit a variety of different body shapes and sizes. In the exemplary configuration of the garment 36 as shown in FIG. 4, the vest 50 may have a first strap 52 and a second strap 54 and the first strap 52 and the second strap 54 may be sized to fit over the shoulder 56 of the patient as shown in FIGS. 4 and 5. The first strap 52 and the second strap 54 may be secured to at least one panel 58, the at least one panel 58 being configured to be secured to a portion of a torso 60 of the patient. The at least one panel 58 may be made from a single component or a variety of different components. It will be understood that the at least one panel 58 may be one singular piece of material or different pieces of material which together comprise the at least one panel 58. As shown in the exemplary embodiment in FIGS. 4 and 5, the at least one panel 58 is made up of different pieces of material.

The first strap 52 may have at least one slider 62 so that the first strap 52 may be adjustable and fit different sized and shapes of patients including their shoulders 56 and torso 60. The at least one slider 62 may be extra-narrow to maximize the adjustment range of the straps. In one embodiment, extra-narrow may be a slider 62 that is less than an inch wide. As shown in FIGS. 4 and 5, the first strap 52 may have one slider 62 where the first strap 52 and the at least one panel 58 meet just below the shoulder 56 and the first strap 52 may have at least one slider 62 on the back portion of the first strap 52 so that the first strap 52 can be easily adjustable from the front and the back of the person wearing the garment 36. In an alternative embodiment, the first strap 52 may have only have one slider 62 on the back of the vest 50 as seen in FIG. 5 and the first strap 52 may be sewn into or otherwise secured or attached to the at least one panel 58 on the front portion of the garment 36. The second strap 54 may also have at least one slider 62 to allow the second strap 54 to be adjustable like the first strap 52. The second strap 54 may have the same configuration of the sliders 60 as the first strap such that the configuration of the sliders 62 is a mirror image of the configuration of the sliders 62 on the first strap 52 and the second strap 54 may have a different configuration of the sliders 62. For example, the second strap 54 may have one slider 62 where the second strap 54 and the at least one panel 58 meet just below the shoulder 56 and the second strap 54 may have at least one slider 62 on the back portion of the second strap 54 so that the second strap 54 can be easily adjustable from the front and the back. In an alternative embodiment, the second strap 54 may have only have one slider 62 on the back of the vest 50 as seen in FIG. 5 and the second strap 54 may be sewn into or otherwise secured or attached to the at least one panel 58.

As shown in FIG. 5, the vest 50 may also include at least one retaining mechanism 64 and the at least one retaining mechanism 64 may include a plurality of slots 66. The retaining mechanism 64 may be a solid piece of material or may be made from various different types of material and/or fabric. The material and/or fabric may have a fusible interfacing to give the retaining mechanism 64 a definite shape and to provide additional support with the retaining mechanism 64. In one embodiment as shown in FIG. 5, the retaining mechanism 64 may be in the shape of a triangle with an elongate portion, but the retaining mechanism 64 can be any variety of different shapes and sizes to accommodate different configurations of straps within the garment 36. As shown in FIG. 5, a portion of the first strap 52 and a portion of the second strap 54 may be inserted into slots 66 in the retaining mechanism 64 so that the portion of the first strap 52 and the second strap 54 that is inserted through each slot in the plurality of slots 66 does not have material or fabric that rubs against the skin of the patient or cause irritation. The first strap 52 and the second strap 54 may also be secured directly into the retaining mechanism 64. For example, the first strap 52 and the second strap 54 may be secured along a central seam in the retaining mechanism rather than the edges of the retaining mechanism 64 to provide easy adjustment for all the straps that are part of the garment 36 and to avoid seams from where the first strap 52 and the second strap 54 are secured to the retaining mechanism 64. Any seams may cause irritation and discomfort against the skin. The retaining mechanism 64 may have one slot 66 that is configured to fit the first strap 52 and the second slot 66 which is configured to fit the second strap 54 within the second slot 66 and inside each slot of the plurality of slots 66 may have a mechanism to retain each strap (which is not visible in the figures) within each slot 66. As a non-limiting example, the mechanism to retain each strap may include a loop and hook fastener, a hook, a clip, or any other type of mechanism to hold the first strap 52 and the second strap 54 in place within the retaining mechanism 64. Alternatively, the first strap 52 and the second strap 54 may be placed through the slot 66 in the retaining mechanism and the material from the first strap 52 and the second strap 54 may come from the slot 66 in the retaining mechanism 64 toward the shoulders 56 of the patient and then form a loop to come over the shoulder and then go back into the at least one slot 66. This configuration of sliders 62 on the third strap 68 and the fourth strap 74 can allow for significant adjustability of the garment 36 and as well as comfort around a variety of different shapes and sizes. The first strap 52 and the second strap 54 may be made from a plush elastic material including a 1.5 inch width fabric so that the first strap 52 and the second strap 54 may comfortably sit against the skin.

Continuing to refer to FIGS. 4 and 5, the vest 50 may include a third strap 68 and the third strap 68 may be configured to engage with different portions of the retaining mechanism 64 through different slots 66. For example, the third strap 68 may be secured with the at least one panel 58. The third strap 68 may have a first portion 70 and a second portion 72. Both the first portion 70 and the second portion 72 of the third strap 68 may be sewn into a portion of the panel at least one panel 58 so that movement of the straps is limited and to secure the third strap 68 to the panel 58 so that the third strap 68 can comfortably sit up against the skin of the patient without causing irritation. The third strap 68 may also be engaged with the retaining mechanism 64 through the plurality of slots 66. In the embodiment shown in FIG. 5, the third strap 68 may pass through the at least one slot 66 of the retaining mechanism 64 and the retaining mechanism 64 may separate the first portion 70 and the second portion 72 of the third strap 68. Alternatively, the first portion 70 and the second portion 72 of the third strap 68 may be fixed on a central seam of the retaining mechanism 64 to maximize the adjustment range of the third strap 68.

Continuing to refer to FIGS. 4 and 5, the vest 50 may also include a fourth strap 74. The third strap 68 may sit across the patient's back at a location that is closer to the patient's shoulder 56 than the fourth strap 74. The fourth strap 74 may also be secured into the at least one panel 58 in the same manner or in a different manner than the third strap 68. The fourth strap 74 may include a first portion 76 and a second portion 78. Both the first portion 76 and the second portion 78 of the fourth strap 74 may be sewn and/or affixed to a portion of the panel 58 so that movement of the straps may be limited and to securely mate the fourth strap 74 with the panel 58 so that the fourth strap 74 can comfortably sit up against the skin of the patient without causing irritation while being secured with the at least one panel 58. The fourth strap 74 may also be engaged with the retaining mechanism 64 through the at least one slot 66. It will be understood that the retaining mechanism 64 may have one slot 66 or more than one slot 66 and each strap may be in contact with the same slot 66 or different slots 66 in the retaining mechanism 64. In one embodiment as shown in FIG. 5 the fourth strap 74 may pass through at least one slot 66 of the retaining mechanism 64. The fourth strap 74 may sit across a portion of the patient's back at a location that is further away from the patient's shoulder 56 than the third strap 68. The retaining mechanism 64 may have one slot 66 or a plurality of slots 66 that are configured retain the number of straps that are used in the garment 36. The third strap 68 and the fourth strap 74 may be made from a plush elastic material including a 1-5 inch width fabric so that the third strap 68 and the fourth strap 74 may comfortably sit against the skin. The third strap 68 and the fourth strap 74 may also include at least one slider 62. As shown in one embodiment in FIG. 5, the first portion 70 and the second portion 72 of the third strap 68 may each have at least one slider 62 and the first portion 76 and the second portion 78 of the fourth strap 74 may also each have at least one slider 62.

Referring to FIGS. 4 and 5, the panel 58 may be made from one material or it may be made from different materials joined and/or secured together to form the panel 58. Additionally, the panel 58 may be made from one component or more than one component that are secured and/or joined together to form the panel 58. The panel 58 may include different mechanisms to secure the vest 50 in place and make it more comfortable and breathable for the patient. The panel 58 may have a first side 80 and a second side 82 opposite the first side 80. The first side 80 of the panel 58 may sit against the skin of the patient and may be made from the same or different material than the second side 82 of the panel 58. In an exemplary embodiment, a first portion 84 of the panel 58 may be on the first side 80 of the panel 58 and may be made out of material including a highly flexible material, such as elastane or another flexible fabric to allow movement of the first portion 84 of the panel 58 when a patient's body moves. The first portion 84 of the panel 58 may be entirely made from the same material or be made from a variety of different materials and the different materials may be sewn or secured together by securing mechanism. The first portion 84 may be made from one segment or more than one segment.

Continuing to refer to FIG. 4, the panel 58 may also have a second portion 86 and the second portion 86 may be entirely made from one material or more than one material. The second portion 86 of the panel 58 may be made from one segment or more than one segment. Each segment may be made from one material or more than one material. As shown in FIG. 4, the first portion 86 of the panel 58 may consist of at least two segments being releasably or permanently coupled by a fastening mechanism 88. The fastening mechanism 88 may be a zipper, including a 5-15 inch length zipper, or any other fastener to secure the vest 50 to the patient. The zipper may be made from metal and/or plastic material. In one exemplary embodiment, the fastening mechanism 88 may be a zipper that is easily accessible from the second side 82. The zipper may be a straight zipper or it may be configured using different angles depending upon the configuration of the garment 36. The fastening mechanism 88 may also include the fastening mechanism 88 on the first side 80 of the panel so that the vest 50 can be secured from the inside on the first side 80. For example, the fastening mechanism 88 on the first side 80 may be a swan hook that is between 0.1-1.5 inches or any other type of fastening mechanism 118. The fastening mechanism 88 on the first side 80 may be, for example, help secure the garment 36 in place when the fastening mechanism 88 on the second side 82 is not fully secured. The second portion 86 of the panel may be made from material or fabric like polyester, viscose, rayon, and spandex and/or a combination of these fabrics. In one example, the material may be a double knit jersey dress fabric to allow for comfort and the elasticity of the vest 50 when it is secured to the patient.

A third portion 90 of the panel 58 may be made from one segment or multiple segments. As shown in FIG. 4, the third portion 90 may have seven different segments. Each of the third portion 90 segments may be made from material or fabric including highly flexible material, such as elastane or another flexible fabric to allow movement of the third portion 90 of the panel 58. The third portion 90 is configured to secure an attachment mechanism 92 (not shown) with at least one segment of the third portion 90. The attachment mechanism 92 may configured to engage with the transmission coil 24 and help to maintain alignment between the transmission coil 24 and the receiving coil 20 so that that receiving coil 20 can be inductively powered through the patient's skin by the transmission coil 24 (as seen in FIG. 2) while the garment 36 maintains the alignment of these two components while the patient moves. In one configuration, the third portion 90 may be made from a material and/or fabric which allows the attachment mechanism 92 to be secured to or through the particular material or fabric so that the attachment mechanism 92 is secured in place.

As shown in the exemplary embodiment in FIG. 4, each segment in the second portion 86 of the panel 58 may be configured in segments to secure the third portion 90 of the panel 58 that is closest to the fastening mechanism 88. Specifically, the second portion may include segments 86ₐ, 86_{b}, 86_{c}, and 86_{d.} and these four segments create an “X” across the upper right-side chest of the patient while the vest 50 is secured to the torso 60. This “X” configuration may secure the third portion 90ₐ in place so that is the third portion 90ₐ is secured to the attachment mechanism 92 and so there will be minimal movement of the third portion 90ₐ to maintain the alignment of the transmission coil 24 and the receiving coil 20 to allow for continuous inductive charging. The segments 86ₐ, 86_{b}, 86_{c}, and 86_{d} may be reinforcement bands linking to the shoulders and other portions of the garment 36 to provide tensioning at the center of the “X”. These reinforced bands may concentrate tension through the middle of the “X” to help the transmission coil 24 deform to the shape of the body when the transmission coil 24 is secured to the garment 36. Also, the stretch in the segments 86ₐ, 86_{b}, 86_{c}, and 86_{d} can help to isolate twisting and other movements of the body. In this embodiment, the transmission coil 24 may be engaged with the attachment mechanism 92 within the vest 50 and the transmission coil 24 may be disposed approximately between 1-5 inches from a center of the vest 50. While the transmission coil 24 is engaged with the attachment mechanism within the vest 50, the transmission coil 24 may move vertically, horizontally, and/or size-to-side up to approximately 3 inches in any direction.

Continuing to refer to FIGS. 4 and 5, a fourth portion 94 of the panel 58 may be made from one segment or multiple segments. As shown in FIG. 4 in one exemplary embodiment, the fourth portion 94 of the panel 58 may have two segments and these two segments may be made from a material or fabric including a power mesh/low stiffness mesh which is a durable fabric that may be have a bidirectional stretch or a single directional stretch and may be made from nylon and other elastic materials that have good recovery properties to help hold the vest 50 in place. Certain lower stiffness zones may isolate the transmission coil 24 from other movements while certain higher stiffness zones may assist in anchoring the transmission coil 24 to selected anatomical reference points. It will be understood throughout the application that “high(er) stiffness zone(s)” and/or “high(er) stiffness material(s)” may be at about >about 2× stiffer than the “low(er) stiffness zone(s)” and/or the “low(er) stiffness material(s).” A fifth portion 96 of the panel 58 may be made from one segment or multiple segments. As shown in FIG. 4 in one exemplary embodiment, the fifth portion 96 of the panel 58 may include two segments and these two segments may be made from a material or fabric including a plush elastic to help hold the vest 50 in place while also being comfortable to wear for long periods of time. For example, the plush elastic may be a fabric with an even pile equivalent or longer and less dense than velvet pile. A sixth portion 98 of the panel 58 may be made from one segment or multiple segments. As shown in FIG. 4, the sixth portion 98 of the panel 58 may have one segment and may be made from material like the dot silicon elastic which is stretchy on the second side 82 of the panel 58 and has silicon dots on the first side 80 of the panel 58 to help keep the vest 50 in place. A seventh portion 100 of the panel 58 may be one segment or multiple segments. As shown in FIG. 5, in one exemplary embodiment, the seventh portion 100 of the panel 58 may have two segments and these two segments may be made from a material or fabric including a stabilized nylon to join the third strap 68 and the fourth strap 74 with the panel 58. As shown in FIG. 4, the panel 58 may be joined to the first strap 52 and the second strap 54 with at least one slider 62 proximate the seventh portion 100. The seventh portion 100 may be a reinforced material and/or fabric to secure the third strap 68 and the fourth strap 74 to the panel 58. An eighth portion 102 of the panel 58 may be made from one segment or multiple segments. As shown in FIG. 4, in one exemplary embodiment, the eighth portion 102 of the panel 58 may have four segments and may be made from material like a fold over elastic that is durable and comfortable when it comes into contact with the skin. As shown in FIG. 4, the eight portion 102 of the panel may come into direct or indirect contact with skin around the shoulder, chest, and underarm which may be sensitive skin and the material used in the eight portion 102 of the panel 58 may be soft and plush to prevent skin irritation and injury. A ninth portion 104 of the panel 58 may be made from one segment or multiple segments. As shown in FIG. 4, the ninth portion 104 of the panel 58 may have one segment and may be made from material like an elastic and the elastic may be 3 inches to give additional durability to the vest 50 while also being comfortable. In general, the garment 36 will generally use a higher stiffness material in the ninth portion 104 of the panel to maintain alignment of the transmission coil 24 and the receiving coil 20 and secure the garment 36 to the body. The various segments within the panel 58 may be secured together with glue or thread, or another adhesive material.

In an exemplary embodiment as shown in FIGS. 4-5, the total length of the first strap 52, shown as A in FIG. 5, may be approximately between 2-10 inches in length with a tolerance of plus or minus 0.5 inches. The total length of the second strap 54, as shown as length B in FIG. 5, may be approximately between 2-10 inches in length with a tolerance of plus or minus 0.5 inches. Additionally, in an exemplary embodiment, a length C of the eighth portion 102 of the panel 58, maybe approximately between 8-20 inches in length with a tolerance of plus or minus 0.5 inches. Also, a length of the fastening mechanism 88, shown as a length D in FIG. 4, may be approximately between 5-15 inches in length with a tolerance of plus or minus 0.5 inches. The length of the fifth portion 96 and the sixth portion 98 of the panel 58, shown as a length E across the torso in FIG. 4, may be approximately between 10-30 inches in total length with a tolerance of plus or minus 0.5 inches. The length of the first portion 76 of the fourth strap 74, shown as length F in FIG. 4, may be approximately between 1-10 inches in length with a tolerance of plus or minus 0.5 inches. The length and width of one segment of the third portion 90a of the panel 58, shown as a length G_{L} and a width G_{w} in FIG. 4, may be the width G_{w} of approximately between 1-6 inches and the length G_{L} of approximately between 2-8 inches, with a tolerance of plus or minus 0.5 inches. The length and width of another segment of the third portion 90 of the panel 58, shown as H_{L} and H_{w} in FIG. 4, may have the width H_{w} as approximately between 1-6 inches and the length H_{L} of approximately between 2-11 inches, with a tolerance of plus or minus 0.5 inches. The attachment mechanism 92 may be secured underneath the third portion 90 of the panel 58 in one location or more than one location on the body. A length I, as shown in FIG. 5, of the seventh portion 100 of the panel 58 maybe approximately between 5-20 inches with a tolerance of plus or minus 0.5 centimeters. A length of the entire fourth strap 74, shown in FIG. 5 as J, may be approximately between 5-15 inches, with a tolerance of plus or minus 0.5 inches. A length of the third strap 68, shown in FIG. 5 as K, may be approximately between 5-15 inches, with a tolerance of plus or minus 0.5 inches. A length of the neck portion of the panel 58 between the fastening mechanism 88 and the slider 62, shown in FIG. 4 as L, may be approximately between 3-12 inches, with a tolerance of plus or minus 0.5 inches.

In FIGS. 4 and 5, exemplary embodiment of the garment 36 is shown as the vest 50 being worn on the torso 60 of the patient. The vest 50 may cover the upper torso from the shoulder 56 to the under-bust of the torso 60. The back of the vest may have a series of adjustable straps, including the first strap 52, the second strap 54, the third strap 68, and the fourth strap 74 to allow adjustability for different body types and as well as to configure the vest 50 so that is can be comfortably worn by the patient. In general, the garment 36 will generally use a higher stiffness material in the second portion 86 of the panel including segment 86a and 86c. Also, a higher stiffness material may be used in segments 86b and 86d to maintain alignment of the transmission coil 24 and the receiving coil 20 and secure the garment 36 to the body.

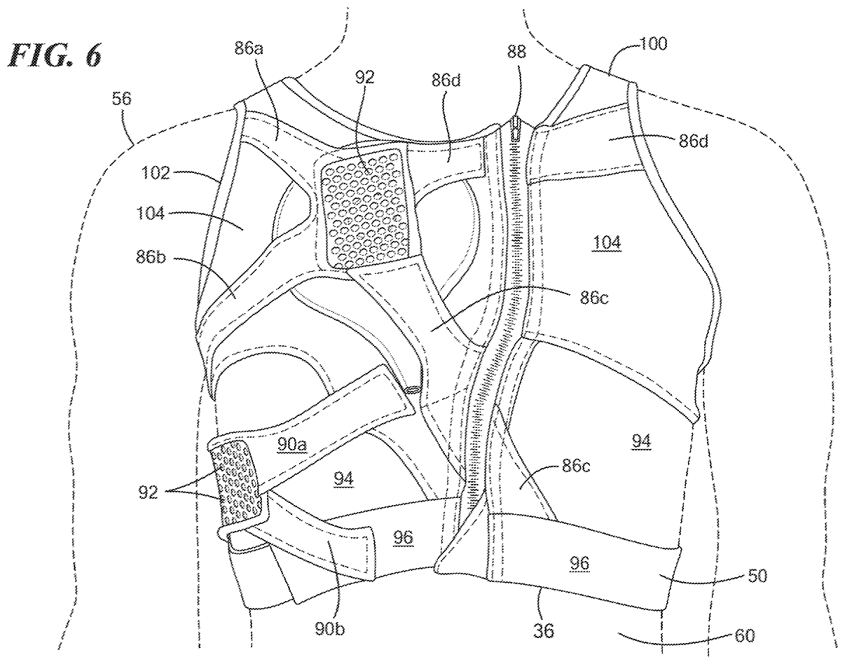

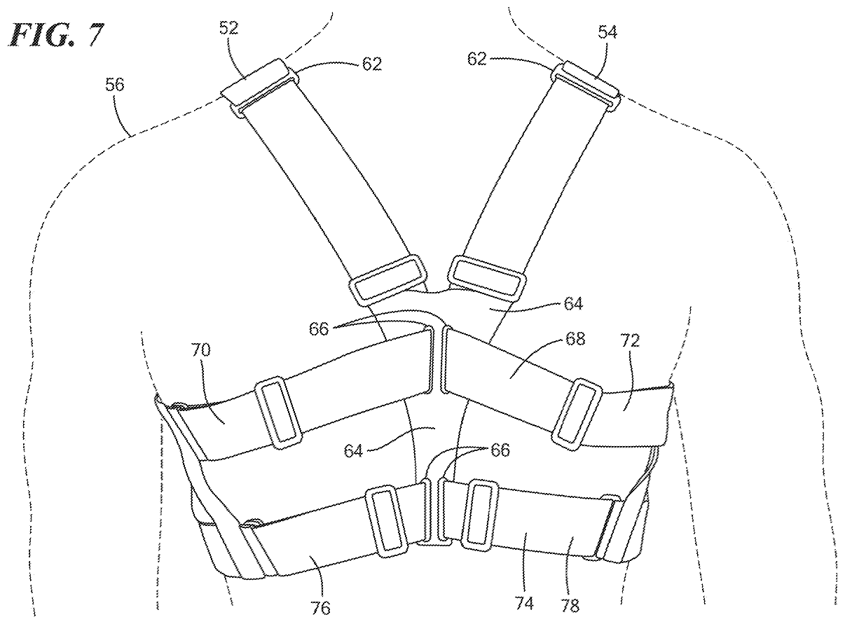

Now referring to FIGS. 6 and 7, is another exemplary embodiment of the garment 36 as a vest 50 being worn on the torso 60 of the patient. In the exemplary configuration of the garment 36 as shown in FIG. 7, the vest 50 may have a first strap 52 and a second strap 54 and the first strap 52 and the second strap 54 may be sized to fit over the shoulder 56 of the patient. The first strap 52 and the second strap 54 may be secured to the at least one panel 58, the at least one panel 58 being configured to be secured to a portion of a torso 60 of the patient. The at least one panel 58 may be made from a single component or a variety of different components which are discussed in more detail. It will be understood that the at least one panel 58 may be one singular piece of material or different pieces of material which together comprise the at least one panel 58.

The first strap 52 may have at least one slider 62 so that the first strap 52 may be adjustable and fit different sized and shapes shoulders 56 and torso 60. As shown in FIGS. 6 and 7, the first strap 52 may have one slider 62 where the first strap 52 and the at least one panel 58 meet just below the shoulder 56 and the second slider 62 near the retaining mechanism 64 so that the first strap 52 can be easily adjusted. The second strap 54 may also have two sliders 62 to allow the second strap 54 to be adjustable like the first strap 52. The second strap 54 may have the same configuration of the sliders 60 as the first strap such that the configuration of the sliders 62 is a mirror image or the configuration of the sliders 62 on the first strap 52 and the second strap 54 may be different configurations of sliders 62. Also, the at least one slider 62 on the first strap 52 and the second strap 54 may also have brightly colored tags so when the garment 36 is secured to the patient's body in the proper manner, the brightly colored tags are visible to alert that the garment 36 is being worn properly. The first strap 52 and the second strap 54 may be sewn into or otherwise secured or attached to the at least one panel 58.

As shown in FIG. 7, the vest 50 may also include the at least one retaining mechanism 64 and the at least one retaining mechanism 64 may include the plurality of slots 66. The retaining mechanism 64 may be a solid piece of material and/or fabric and the fabric may have a fusible interfacing to give the retaining mechanism 64 a definite shape and to provide additional support for the retaining mechanism 64. In the embodiment shown in FIG. 7, the retaining mechanism 64 may be in the shape of a triangle with an elongate portion, but the retaining mechanism 64 can be any variety of different shapes and sizes to accommodate different configurations of straps within the garment 36. A portion of the first strap 52 and a portion of the second strap 54 may be inserted into slots 66 in the retaining mechanism 64 so that the portion of the first strap 52 and the second strap 54 that is inserted through each slot in the plurality of slots 66 does not have material or fabric that may rub against the skin of the patient or cause irritation. The first strap 52 and the second strap 54 may also be secured directly into the retaining mechanism 64. For example, the first strap 52 and the second strap 54 may be secured along a central seam in the retaining mechanism rather than the edges of the retaining mechanism 64 to provide easy adjustment for all the straps that are part of the garment 36. The retaining mechanism 64 may have one slot 66 that is configured to fit the first strap 52 and the second slot 66 which is configured to fit the second strap 54 within the second slot 66 and inside each slot of the plurality of slots 66 may have a mechanism to retain each strap (which is not visible in the figures) within each slot 66 such as a hook and loop fastener or another type of mechanism. Alternatively, the first strap 52 and the second strap 54 may be placed through the slot 66 in the retaining mechanism and the material from the first strap 52 and the second strap 54 may come from the slot 66 in the retaining mechanism 64 toward the shoulders 56 of the patient and then form a loop to come over the shoulder and then go back into the at least one slot 66. This configuration of sliders 62 on the third strap 68 and the fourth strap 74 can allow for significant adjustability of the garment 36 and as well as comfort around a variety of different shapes and sizes.

Continuing to refer to FIGS. 6 and 7, the vest 50 in this configuration may include the third strap 68 and the third strap 68 may be configured to engage with different portions of the retaining mechanism 64 through different slots 66. The third strap 68 may have the first portion 70 and the second portion 72. Both the first portion 70 and the second portion 72 of the third strap 68 may be sewn into a portion of the at least one panel 58 so that movement of the straps is limited and to secure the third strap 68 to the panel 58. The third strap 68 may also be engaged with the retaining mechanism 64 through the plurality of slots 66. In the embodiment shown in FIG. 7, the third strap 68 may pass through the at least one slot 66 of the retaining mechanism 64 and the retaining mechanism 64 may separate the first portion 70 and the second portion 72 of the third strap 68. Alternatively, the first portion 70 and the second portion 72 of the third strap 68 may be fixed on a central seam of the retaining mechanism 64.

Continuing to refer to FIG. 7, the vest 50 may also include the fourth strap 74. The fourth strap 74 may be secured into the at least one panel 58 in the same manner or in a different manner than the third strap 68. The fourth strap 74 may include the first portion 76 and the second portion 78. Both the first portion 76 and the second portion 78 of the fourth strap 74 may be sewn and/or affixed into a portion of the panel 58. The fourth strap 74 may also be engaged with the retaining mechanism 64 through the at least one slot 66. In one embodiment as shown in FIG. 7, the fourth strap 74 may pass through at least one slot 66 of the retaining mechanism 64. The third strap 68 and the fourth strap 74 may also include at least one slider 62. As shown in one embodiment in FIG. 7, the first portion 70 and the second portion 72 of the third strap 68 may each have at least one slider 62 and the first portion 76 and the second portion 78 of the fourth strap 74 may also each have at least one slider 62. The first strap 52, second strap 54, third strap 68, and fourth strap 74 may all be elasticated to distribute tension of the garment 36 around the body and improve the comfort of the garment 36.

Referring to FIG. 6, the panel 58 may be made from one material or it may be made from different materials joined to form the panel 58. Additionally, the panel 58 may be made from one component or more than one component that are secured together to form the panel 58. The panel 58 may include different mechanisms to secure the vest 50 in place and make it more comfortable and breathable for the patient. The panel 58 may include the first side 80 and the second side 82 opposite the first side 80. The first side 80 of the panel 58 may sit against the skin of the patient and may be made from the same or different material than the second side 82 of the panel 58. In the exemplary embodiment, the first portion 84 of the panel 58 may be on the first side 80 of the panel 58 and may be made out of material including a highly flexible material, such as elastane or another flexible fabric to allow movement of the first portion 84 of the panel 58 when a patient's body moves. The first portion 84 may be made from aa material or fabric that lines the garment 36 to make it comfortable as it sits against the skin. The first portion 84 of the panel 58 may be entirely made from the same material or a be made from a variety of different materials and the different materials may be sewn or secured together by securing mechanism. The first portion 84 may be made from one segment or more than one segment.

Continuing to refer to FIG. 6, the panel 58 may also have the second portion 86 and the second portion 86 may be entirely made from one material or more than one material. The second portion 86 of the panel 58 may be made from one segment or more than one segment. As shown in FIG. 6, the first portion 86 of the panel 58 may consist of various segments being releasably or permanently coupled by the fastening mechanism 88. In this exemplary embodiment, the first portion 86 segments may form an “X” with each segment, 86ₐ, 86_{b}, 86_{c}, 86_{d}, and 86ₑ securing the attachment mechanism 92 in place. In one embodiment, segments 86ₐ, 86_{b}, 86_{c}, 86_{d}, and 86ₑ may be under tension when the garment 36 is worn by the patient so that the transmission coil 24 is secured against the body and does not move. The attachment mechanism 92 may include a thin polymer sheet to minimize bulk when wearing the garment 36 and the attachment mechanism 92 may include a plurality of apertures that can allow for adjustment of the receiving coil 24 into a variety of different positions and location. In one exemplary embodiment, the transmission coil 24 may be generally 1-8 inches from the center of the garment when it is secured with the attachment mechanism 92 and the garment 36. Each of the second portion 86 segments may be bands that are pre-tensioned relative to the rest of the garment 36 to prevent movement of the attachment mechanism and/or the transmission coil 24 when there is movement of the body in various directions. The transmission coil 24 may be secured in place by segments 86ₐ, 86_{b}, 86_{c}, and 86_{d} which may be connected to the shoulders, the third strap 68 and the fourth strap 74, as well as the center portion of the panel 58. Any of the segments in the second portion 86 may be sewn down on either side of the fastening mechanism so that the fastening mechanism 88 may be easily secured and unsecured. The vest 50 may have the fastening mechanism 88 on the first side 80 of the panel so that the vest 50 can be secured from the inside as well as the fastening mechanism on the second side 82 of the panel. The fastening mechanism 88 in an exemplary configuration may not be centered on the vest 50 to make space for various segments of the second portion 86 and the third portion 90. When the transmission coil 24 is secured in the attachment mechanism 92 and the garment 36, the transmission coil 24 may move between 1-6 inches in a vertical direction depending upon the height and bust of the patient wearing the garment 36.

The third portion 90 of the panel 58 may be made from one segment or multiple segments. As shown in FIG. 6, the third portion 90 may have different segments. Each of the third portion 90 segments may be made from a reinforced material and/or fabric. The third portion 90 is configured to secure the attachment mechanism 92 to the side of the body, for example in the axilla region. For example, as shown in one configuration, third portion 90ₐ may be a pretensioned band that is sewn down to material and/or fabric near the fastening mechanism 88 third portion 90_{b} may be secured near the base of the vest 50. The third portion 90 may also include segments 90_{c} and 90_{d} (not shown) which form an “X” with the attachment mechanism in a similar configuration as seen with 86ₐ, 86_{b}, 86_{c}, and 86_{d} to secure the transmission coil 24. In one embodiment, segments 90ₐ, 90_{b}, 90_{c}, and 90_{d} may be under tension when the garment 36 is worn by the patient so that the transmission coil 24 is secured against the body and does not move. In one embodiment, the attachment mechanism 92 may be made from a flexible polymer to provide comfort and robustness when the transmission coil 24 is secured to the attachment mechanism 92. When the transmission coil 24 is secured to the attachment mechanism 92 on the side of the body within the garment, the transmission coil 24 may be able to move approximately between 1-8 inches in a horizontal direction to account differences in surgical placement of the receiving coil 20.

Continuing to refer to FIG. 6 a fourth portion 94 of the panel 58 may be made from one segment or multiple segments. As shown in FIG. 4 in one exemplary embodiment, the fourth portion 94 of the panel 58 may have two segments and these two segments may be made from a material or fabric including a power mesh/low stiffness mesh which is a durable fabric that may be have a bidirectional stretch or a single directional stretch and may be made from nylon and other elastic materials that have good recovery properties to help hold the vest 50 in place. A fifth portion 96 of the panel 58 may be made from one segment or multiple segments. As shown in FIG. 6 in one exemplary embodiment, the fifth portion 96 of the panel 58 may include two segments and these two segments may be made from a material or fabric including a plush elastic to help hold the vest 50 in place while also being comfortable to wear for long periods of time. The fifth portion 96 may be configured to sit just under the base of the sternum and clear the breast/pectoral region of the body. Also, as shown in FIG. 6, a portion of the third portion 90_{b} may be secured to a portion of the fifth portion 96. In general, lower stiffness materials may be used over the breast area of the patient in any embodiment which may provide increased comfort over and around the breast tissue.

A seventh portion 100 of the panel 58 may be one segment or multiple segments. As shown in the configuration in FIG. 6, in one exemplary embodiment, the seventh portion 100 of the panel 58 may have two segments and these two segments may be made from a material or fabric including a stabilized nylon to join the first strap 52 and the second strap 54 with the panel 58. The seventh portion 100 may be made from a material or fabric that can stretch at the shoulders to isolate shoulder movement, while controlling tension levels within the garment 36 to minimize movement of the transmission coil 24. An eighth portion 102 of the panel 58 may be made from one segment or multiple segments. As shown in FIG. 4, in one exemplary embodiment, the eighth portion 102 of the panel 58 may have four segments and may be made from material like a fold over elastic that is durable and comfortable when it comes into contact with the skin. As shown in FIG. 6, the eight portion 102 of the panel may come into contact with skin around the shoulder, chest, and underarm which may be sensitive and the material used in the eight portion 102 of the panel 58 may be soft and plush to try and prevent skin irritation. A ninth portion 104 of the panel 58 may be made from one segment or multiple segments. As shown in FIG. 4, the ninth portion 104 of the panel 58 may have multiple segments and may be made from material or fabric to help secure the garment 36 in place. The ninth portion 104 may be made from a lightweight mesh to allow for a larger range of movement within the torso and provide breathability and cooling while the garment 36 is being worn. Additionally, any portion of the garment 36 may be made from a lightweight and/or wicking fabric to help keep the skin cool and dry while the garment is being worn.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

1. An alignment vest for holding an external coil in a predetermined location, the alignment vest comprising:
at least one attachment mechanism for holding the external coil in the predetermined location;
a panel secured to the attachment mechanism; and
a plurality of straps, the panel being secured to at least one of the plurality of straps.

2. The alignment vest of claim 1, wherein the at least one attachment mechanism comprises a first attachment mechanism and a second attachment mechanism, the first attachment mechanism and the second attachment mechanism being secured to at least a portion of the panel.

3. The alignment vest of claim 2, further comprising a first band, a second band, a third band, and a fourth band, the first band, the second band, the third band and the fourth band each being pretensioned bands that are configured to secure the first attachment mechanism to the panel.

4. The alignment vest of claim 3, further comprising a fifth band, a sixth band, a seventh band, and an eighth band, the fifth band, the sixth band, the seventh band, and the eighth band each being pretensioned bands that are configured to secure the second attachment mechanism to the panel.

5. The alignment vest of claim 1, wherein the panel further comprises a plurality of portions and a fastening mechanism, the fastening mechanism being a zipper and the plurality of portions being releasably secured together with the zipper.

6. The alignment vest of claim 1, wherein the plurality of straps includes a first strap, a second strap, a third strap, and a fourth strap, at least a portion of each strap being secured to a portion of the panel.

7. The alignment vest of claim 6, wherein each strap from the plurality of straps has at least one slider, the at least one slider being configured to adjust a length of the strap.

8. The alignment vest of claim 7, wherein the at least one slider is made from a non-conductive material.

9. The alignment vest of claim 7, wherein at least a portion of the first strap, the second strap, the third strap, and the fourth strap are secured to a retaining mechanism.

10. An alignment vest for holding an external coil in a predetermined location, the alignment vest comprising:
at least one attachment mechanism for holding the external coil in the predetermined location;
a panel including a fastening mechanism, a first band, a second band, a third band, and a fourth band, the first band, the second band, the third band and the fourth band each being pretensioned bands that are configured to secure the at least one attachment mechanism to the panel; and
a plurality of straps, the panel being secured to at least one strap of the plurality of straps.

11. The alignment vest of claim 10, wherein the attachment mechanism includes a first attachment mechanism, the alignment vest further comprising a second attachment mechanism, a fifth band, a sixth band, a seventh band and an eighth band, the fifth band, the sixth band, the seventh band and the eighth band each being pretensioned bands that are configured to secure the second attachment mechanism to the panel.

12. The alignment vest of claim 10, wherein the fastening mechanism is at least one of a zipper or a swan hook.

13. The alignment vest of claim 12, wherein the fastening mechanism is the zipper, and wherein the panel has at least two portions, the at least two portions being releasably secured together with the zipper.

14. The alignment vest of claim 13, wherein the zipper is made from a non-conductive material.

15. An alignment vest for holding an external coil in a predetermined location, the alignment vest comprising:
a panel including a fastening mechanism;
a first attachment mechanism for holding the external coil in a first predetermined location, the first attachment mechanism being secured to a portion of the panel;
a second attachment mechanism for holding the external coil in a second predetermined location, the second attachment mechanism being secured to a portion of the panel; and
a first strap, a second strap, a third strap and a fourth strap, at least a portion of the panel being secured to at least a portion of the first strap, the second strap, the third strap and the fourth strap.

16. The alignment vest of claim 15, wherein the fastening mechanism includes a zipper.

17. The alignment vest of claim 15, wherein at least one of the first strap, the second strap, the third strap, or the fourth strap includes a slider configured to adjust a length of the respective strap.
